(19) United States
(12) Patent Application Publication
(10) Pub. No.: US 2016/0021889 A1
(43) Pub. Date: Jan. 28, 2016

(54) Treatment Liquid for Cleaning an Implant Part

(71) Applicants: Markus SCHLEE, (US); Urs BRODBECK, (US); Holger ZIPPRICH, (US)

(72) Inventors: Urs Brodbeck, Erlenbach (CH); Holger Zipprich, Malchen (DE)

(21) Appl. No.: 14/765,554

(22) PCT Filed: Feb. 5, 2014
(86) PCT No.: PCT/EP2014/052270

§ 371 Date: Aug. 3, 2015

(30) Foreign Application Priority Data
Feb. 5, 2013 (DE) 10 2013 201 883.5

(51) Int. Cl. A01N 59/12; A01N 37/36

(52) U.S. Cl. CPC A01N 59/12; A01N 37/36

(57) ABSTRACT

A treatment liquid shall be suitably designed specifically for cleaning bacterially contaminated surfaces of bone implants, dental implants or other components contaminated with a biofilm. For this purpose, the treatment liquid according to the invention comprises, as one of its basic constituents, an aqueous solution of an acid which is provided with a metallic salt in such a manner that a conductivity of at least 30 mS/cm, preferably at least 75 mS/cm, and particularly preferably at least 150 mS/cm results.

The invention relates to a treatment liquid, in particular for cleaning bacterially contaminated surfaces of bone implants, dental implants or other components contaminated with a biofilm. It also relates to the use of the treatment liquid, in particular for cleaning a dental-implant part anchored in the jawbone of a patient.

A treatment element, in particular for use with an implant part, as well as a method for cleaning a dental-implant part, are known from the German patent application with the reference number 10 2012 022 593.8, not prior published, whose entire disclosure is incorporated by reference. In fact, a biofilm may form on the firm surface of implants, enclosed by tissue and tissue liquid, which biofilm is colonized by bacteria which may finally lead to chronic and recurrent infections. This syndrome is called periimplantitis. In particular in the dental area, similar to parodontitis, a combination of neglected mouth hygiene, adhesion of a biofilm on the usually microrough surface of the post part, and other factors lead to the full picture of periimplantitis, which is characterized by an increasing charge and destruction of the hard and soft tissues. The areas where the hard and/or soft tissues retreat are usually covered by a biofilm.

The cleaning method described in the above-mentioned application is based on the concept, to kill and remove the biofilm or the germs forming the contamination, starting from the implant surface, without damaging the implant surface. For this purpose, an electrolytic process is provided, by which the ions (cations and/or anions) are conveyed by means of electrostatic forces through the biofilm. These ions react chemically or electrochemically on the implant surface. Through these reactions, new compositions of matter are created and/or the ions themselves and/or parts of these ions are converted into the atomic state. Furthermore, it is also possible that the ions react with the surface material (e.g. development of an oxide layer or erosion of material).

The germicidal effect of this process is based on different effects. On the one hand, ions from the biofilm itself (and also from the bacteria) are transported to the anode or cathode through the application of an electric voltage. This may lead to a killing of bacteria and viruses. Furthermore, the ions, while passing through the biofilm, may undergo biochemical reactions, which may also lead to a killing of bacteria and/or viruses. Another possibility of killing consists in that the compositions of matter newly formed on the implant surface possess an antibacterial and/or antiviral and/or antifungal effect. This may, of course, also happen when the ions are converted into the atomic state. All of the above-described processes entail in general the risk that toxic, cytopathic and/or bone-damaging substances or compositions of matter might be formed or released.

The present invention is based on the problem to provide a treatment liquid which is particularly suitable for use in the above-mentioned treatment system. In particular, a treatment liquid shall be provided which is particularly and with high efficiency suitable for treating or cleaning components of a dental-implant system, of other implant parts, such as, for example, bone implants in general, or possibly also other component parts covered with a biofilm.

This problem is solved according to the invention by forming the treatment liquid as the basic constituent of an aqueous solution of an acid mixed with a metallic salt in such a manner that a conductivity of at least 30 mS/cm, preferably at least 75 mS/cm, and particularly preferably at least 150 mS/cm, results.

Advantageous embodiments of the invention are the subject matter of the dependent claims. Further and/or alternative advantageous embodiments of the invention are also obvious from the description of the figures.

The invention starts out on the consideration that a suitable treatment liquid should be specifically designed, on the one hand, for utilizing and promoting the effects based on an electrolytic cleaning of the above-mentioned system, while, on the other hand, especially in view of an imaginable use of the liquid also for cleaning implant parts in the inserted state, the formation of toxic, cytopathic and/or bone-damaging substances or substances which are generally hazardous to health should consistently be avoided or at least be reduced to a clinically acceptable level. To make this possible, the treatment liquid is specifically designed, on the one hand, for a suitability as electrolyte for the above-mentioned electrochemical processes, a conductivity suitably chosen for this purpose being in particular provided. On the other hand, however, the choice and composition of the basic constituents of the treatment liquid is also chosen in such a way that the substances present in the liquid as well as the products generated in the course of the electrochemical processes particularly favor the killing of germs and possibly also the mechanical detachment of the biofilm, without causing, in return, any considerable damages or hazards for the body tissue or bone tissue.

Advantageously, a salt of aluminium, of an alkali metal or of an alkaline earth metal, in particular a potassium, sodium, magnesium, or calcium salt is provided as metallic salt. A potassium, or sodium salt is most particularly preferred. Furthermore, chlorine or iodine is advantageously provided as a halogen for the metallic salt. The acid is particularly preferably an organic acid, particularly preferably an α-hydroxy carbonic acid, preferably lactic acid, citric acid, ethanoic acid or malic acid, or a combination of these constituents. A particularly advantageous composition of the basic constituents will be given if—for the sake of pH-value buffering for compensating a hydroxide production, as required—the pH-value of the treatment liquid is less than 5, preferably less than 4, particularly preferably approx. 2.7 to 2.9.

In a particularly advantageous and in an independently inventive manner, the treatment liquid is used for cleaning bacterially contaminated surfaces of bone implants, dental implants or other components contaminated with a biofilm, i.e. in general for removing a biofilm.

The advantages achieved with the invention consist in particular in the fact that through the combination of the basic constituents metallic salt and acid and the resulting electric conductivity due to the ion density, the desired electrolytic or electrochemical processes taking place during cleaning can be particularly promoted. In particular, the acid can counteract, in the manner of a pH-value buffering, an undesired increase of the pH-value as a consequence of the generation of metal hydroxide caused by the reaction.

An exemplary embodiment of the invention is explained in detail by means of a drawing, in which

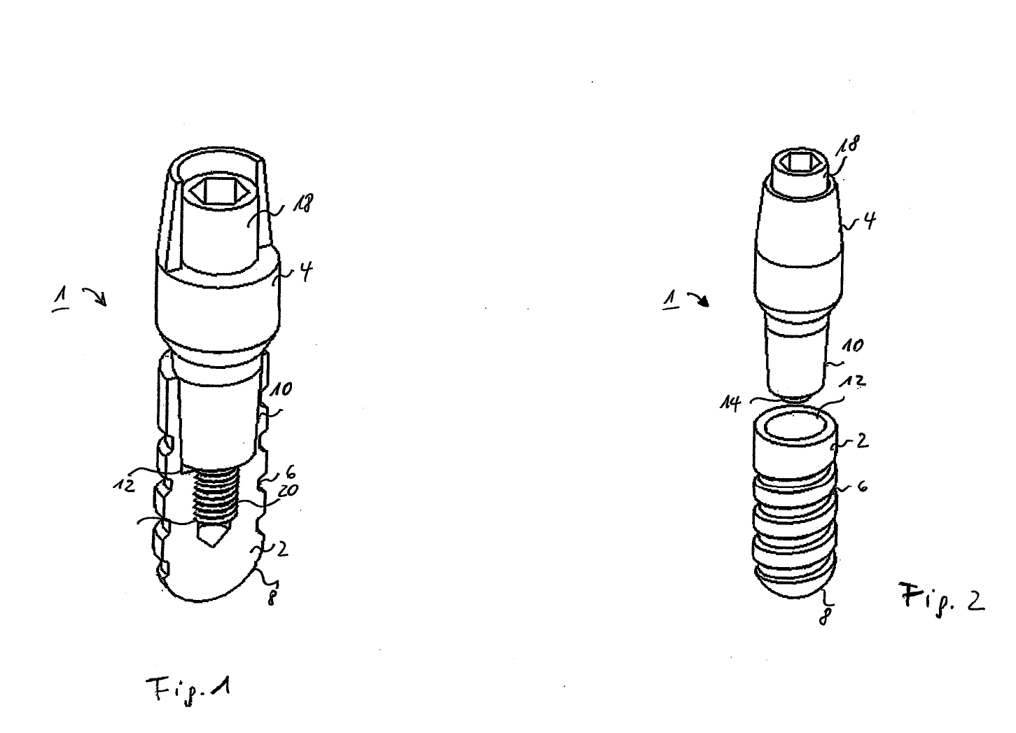

FIG. 1 shows a two-part dental-implant system in mounted state,

FIG. 2 is an exploded view of the dental-implant system according to FIG. 1,

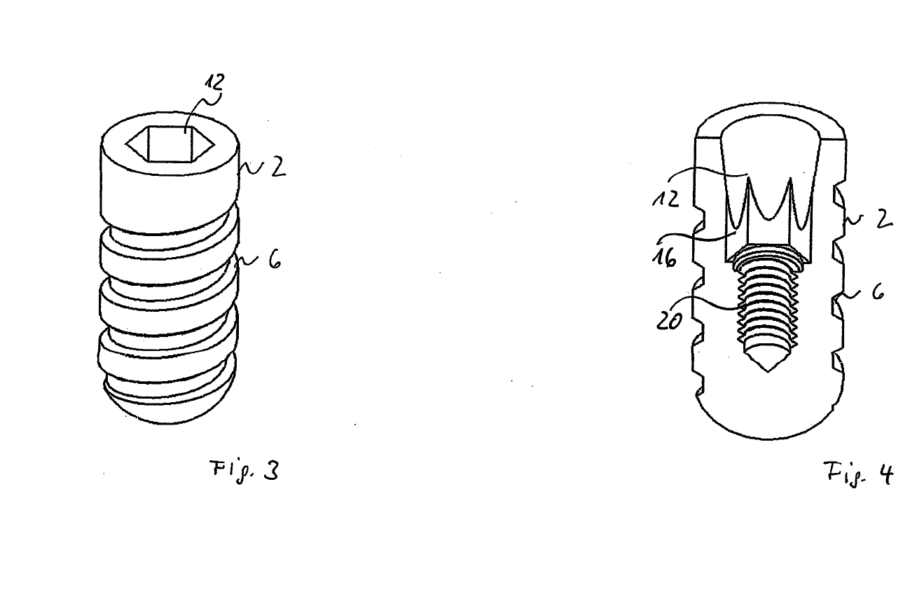

FIG. 3 is a lateral view of the post part of the dental-implant system according to FIG. 1, 2,

FIG. 4 is a longitudinal sectional view of the post part according to FIG. 3,

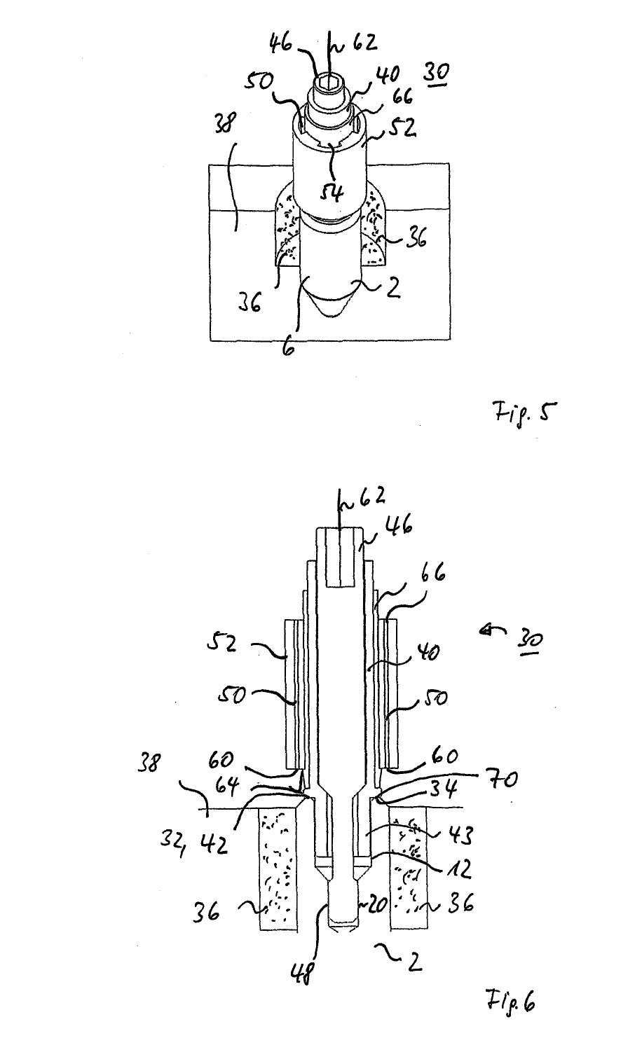

FIG. 5 is a perspective view of a treatment element for the post part according to FIG. 3, 4,

FIG. 6 is a longitudinal sectional view of the treatment element according to FIG. 5,

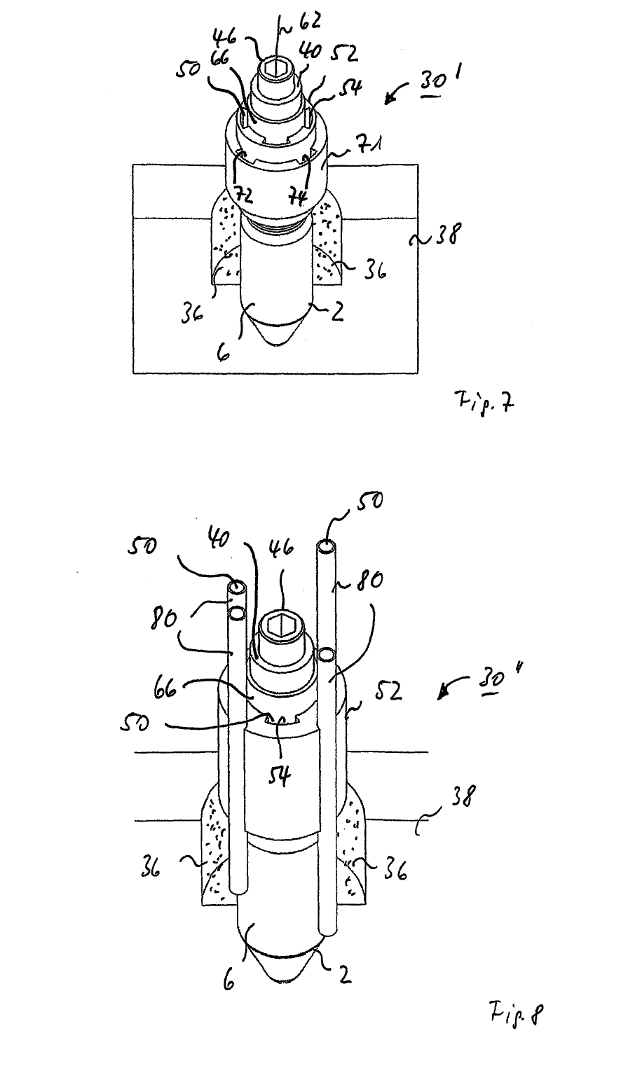

FIG. 7, 8 are each a perspective view an alternative embodiment of a treatment element,

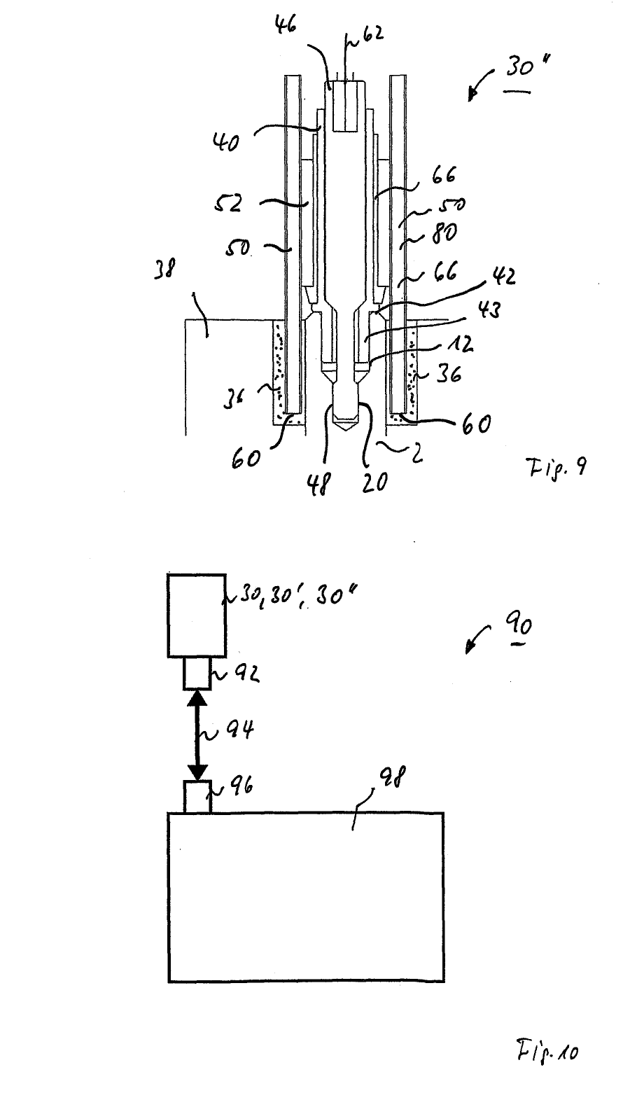

FIG. 9 is a longitudinal sectional view of the treatment element according to FIG. 8, and

FIG. 10 shows a treatment system.

Identical parts are marked with the same reference numbers in all figures.

The treatment liquid according to the invention is in the following explained in detail by means of a use in a treatment system for a dental implant, considered as a preferred use, it is, however, not limited in any way to this use. Rather is an application for treating or cleaning other components of a dental-implant system, other implant parts, such as, for example, bone implants in general, or possibly also other component parts covered with a biofilm analogously considered.

The dental-implant system 1 according to FIG. 1 is intended for use in the jawbone in the place of an extracted or shed tooth, to fix there a prosthetic part or a crown serving as a denture piece. The dental-implant system 1 is made up of several parts and comprises a first implant part 2 configured as a so-called post part, and a second implant part 4, also referred to as superstructure part or abutment, associated therewith and provided for fastening a denture piece. The first implant part 2 or post part is provided on its outside with an external thread 6, configured, in particular at the apical end 8, as a self-cutting screw thread, with which the first implant part 2 or post part can be inserted in the intended place in the jawbone by screwing it in.

In order to make it possible, after suitably fastening the denture piece or the prosthesis on the abutment or second implant part 4, to anchor it in the post part or first implant part 2 with high mechanical stability, a connection stud 10 is moulded onto the second implant part 4, which connection stud 10 can be pushed into an associated receiving duct 12 provided in the first implant part 2. By pushing the connection stud 10 into the receiving duct 12, the implant parts 2, 4 are mechanically coupled with each other. To ensure a high mechanical stability, the outer contour of the connection stud 10 is adapted to the inner contour of the receiving duct 12, it being possible that both of them are of conical shape, viewed in longitudinal direction (exemplary embodiment according to FIG. 2). Furthermore, as provided in particular in the exemplary embodiment according to FIG. 3, the outer contour of the connection stud 10—and in according adaptation, also the inner contour of the receiving duct 12—can be provided in cross-section with a multiple symmetry (in the exemplary embodiment, a sixtuple symmetry), so that, when joining the above-mentioned components, a rotational locking gear is created and, thus, a reliable rotational orientation of the abutment relative to the post part can be adjusted. In the exemplary embodiment according to FIG. 3, 4, an indexing element 14, whose cross-section also shows a multiple symmetry, is arranged on the end-side of the connection stud 10 for the purpose of an indexing or for creating a rotational locking gear, said indexing element 14 engaging in assembled condition into a corresponding associated duct end piece 16 in the receiving duct 12.

In the exemplary embodiment, the dental-implant system 1 is configured for a screw connection of the implant parts 2, 4 with each other. For this purpose, a connecting screw 18 is provided, which engages into a screw thread 20 provided inside the first implant part 2. With regard to the choice of their materials, the implant parts 2, 4 are suitably adapted to the intended application and are generally made of a ceramic material, such as, for example, zirconium oxide or aluminium oxide, or else of a suitably chosen metal, such as, for example, titanium.

In general, dental-implant systems, in particular also two-part implant systems of the above-described type, present the problem that inflammations or inflammation focuses may arise due to a penetration of bacteria or germs into the tissue area near the place of insertion, in particular in the area of the external thread 6 cut into the jaw. Such inflammations, in particular also as a consequence of a so-called periimplantitis, may lead to a serious deterioration of the tissue and the bone in the area of the place of insertion, especially when they are able to develop and take hold over a long period. Without suitable countermeasures, these deteriorations may lead to the necessity to remove the entire implant system, i.e. in particular also the already inserted post part or second implant part 4, from the bone and replace it by another prosthetics. This most undesirable effect caused by the periimplantitis may, therefore, lead to a total loss of the implant system, so that renewed surgical measures, such as, for example, scraping out the afflicted area in the jawbone and treatment with a new implant system might become necessary. Such a removal may, furthermore, entail a loss of bone or other loss of tissue substance, which in the extreme case may even make a new treatment with another implant completely impossible. Such a necessity of a new treatment caused by a periimplantitis may occur even after relatively long periods after the first insertion of the implant system of, for example, up to several years or even decades.

The germs or bacteria observed in connection with a periimplantitis may in principle colonize the inside of the post part 2, but, as a rule, they preferably adhere directly on the surface of the post part 2 inserted into the jawbone, in the contact area with the surrounding tissue or bone material, i.e. in particular in the area of the external thread 6. In the area of the latter, the surface of the post part 2 can be provided with a roughening or the like, in order to particularly promote the growing-in of the tissue or the bone and to support the healing-in of the post part 2 after its insertion. Especially in the area of such a roughening of the surface, actually considered as particularly favorable for the implant system, however, the colonization of germs or bacteria may take place increasedly, the roughness making a specific removal of the existing germs or bacteria even more difficult.

Therefore, suitable countermeasures are urgently required, in order to be able, in case of a beginning or already existing periimplantitis and under preservation of the already inserted implant system, i.e. in particular of the already inserted post part 2, to efficiently combat the inflammation focus and to kill the germs that have penetrated, so that afterwards, sound tissue or sound bone substance can develop again in the area around the external thread 6. For this purpose, it is desirable, in addition to a specific killing of the germs or bacteria in the afflicted area, to also reliably remove their material residues and fragments from the space area concerned, so that then, the afflicted area can be filled again by sound tissue or bone material and an intimate connection between the outer surface of the post part 2 and the surrounding tissue or bone material can develop again. In addition, the biofilm formed by the bacteria layer, including the organic residues of killed bacteria, should reliably be removed.

For this purpose, i.e. for killing germs or bacteria in the insertion area of the post part 2 and in particular also for subsequently rinsing, removing and carrying away the residues of tissue and material of the killed bacteria, a treatment element 30 is provided, like the one shown in FIG. 5 in a perspective view and in FIG. 6, in a longitudinal section. In the exemplary embodiment, the treatment element 30 is designed, due to actually two-part embodiment of the implant system 1, in the manner of a treatment abutment, and is provided for the shown two-part implant system 1 for carrying out the before-mentioned treatment, wherein the treatment abutment 30 should temporarily be placed on the post part 2 in the place of the actual abutment or second implant part 4. Therefore, the following embodiments refer to this case of a two-part implant system 1; but, of course, in an analog embodiment, a corresponding use for single-part implants may also be provided; for this purpose, it would just be necessary to suitably configure the mechanical connection of the treatment element 30 with that part of the implant system which remains in the jawbone during the treatment, for example via a suitable contact surface, with which the treatment element 30 can be placed onto the abutment of the implant in the place of the prosthetics. Alternatively, the treatment element 30 can be placed on top of the actual abutment 4 of the implant system 1, so that a use, for example, for combating an inflammation of the soft tissue (mucositis) through killing of the bacteria and cleaning the surface can be provided, without having to remove the actual abutment 4 for that purpose.

With the two-part embodiment of the implant system 1 provided in the exemplary embodiment, first of all—possibly after removal of the prosthetics fixed on the actual abutment or second implant part 4—the screw connection between the first and second implant parts 2, 4 is detached and the second implant part 4 is taken off, for carrying out treatment described in detail in the following. The first implant part or post part 2 remains in the jawbone. Then, the treatment abutment 30 is placed onto the post part 2 instead of the actual abutment 4 and connected with the latter via the screw connection. For this purpose, the treatment implant 30 includes a substantially planar contact surface 32 auf, with which it can be placed onto the end edge 34 of the post part 2. The contact surface 32 may under certain circumstances also fulfil the function of a sealing face and be designed accordingly; in particular, it can be of a conical design for that purpose.

With regard to its design and fundamental configuration, the treatment abutment 30 is based on the main concept of specifically killing the germs or bacteria present in the insertion area of the post part 2 through specifically feeding a cleansing agent or disinfectant, whereby any residues or fragments of germs and/or bacteria still adhering on the surface of the post part 2, in particular in the area of the external thread 6, shall be detached from the outer surface of the post part 2 through a suitable charging with current or current impulses, so that such residues can then be washed out.

In a first aspect, which is independently considered as inventive both with regard to the configuration of the system and with regard to the provided steps of the treatment method, the treatment element 30 is, therefore, designed, both structurally and functionally/conceptually, for specifically feeding a treatment liquid for killing the germs or bacteria and/or for cleaning the inserted implant part 2 into the insertion area of the post part 2, in particular the area of the latter's external thread 6.

In a second aspect, which is also independently inventive both with regard to the configuration of the system and the choice and composition of the basic constituents of the utilized treatment liquid and with regard to the provided steps of the treatment method, the treatment element 30 is designed for reliably detaching the killed bacteria or germs, respectively their residues or fragments, from the outer surface of the post part 2, so that they can then be washed out and, afterwards, sound tissue or bone material can again get into contact with the surface of the post part 2 and the latter can again grow completely into sound tissue or bone material. For detaching the bacteria or germs, respectively their residues or fragments, from the surface, it is provided to wet the latter with a conductive treatment liquid, charging it with current, possibly in the form of pulsed current impulses. It has also turned out most surprisingly that exactly this charging with current, in combination with suitably chosen ion concentrations in the treatment liquid, seems to effect the detachment of the bacteria or germs, respectively their residues or fragments, from the surface underneath in a particularly reliable manner, even if said surface is roughened and, in fact, particularly promotes the adhesion of organic material due to its surface structure.

This is based on the surprising discovery that the charging of the post part 2 with current, using a suitably chosen treatment liquid, in the area of the outer surface of the post part, i.e. in particular in the area of the external thread 6, leads to an electrolytic reaction in the treatment liquid and, thus, possibly to the generation of gas bubbles in the immediate vicinity of the surface. Through this formation of gas bubbles on the surface of the post part 2, the superficially adhering components or fragments of the germs or bacteria are also detached and completely removed, so that they cannot offer a basis or a nutrient medium for a new colonization of germs in these areas. What remains is a roughened and porous surface, cleaned from germs, bacteria or their components or residues, of the post part 2, which can serve well as a basis for a future integration into the regrowing bone tissue. The remaining surface can also be formed by a titanium-oxide layer, which would also arise when anodizing the surface.

A particular promotion of this separation of superficially adhering biofilm components from the inserted post part 2, which is desirable in the sense of a reliable cleaning of the surface, can be achieved through an advantageous, particularly well suited process guidance during the charging with current. Said process guidance can be such that due to the current flow, an electrolytic formation of gas bubbles taking place in the area of the inserted surface is particularly increased. Here, the post part 2 can be switched anodically or cathodically. In particular in case of an at least temporary cathodic switching of the post part 2, hydrogen gas, which contributes in a particularly efficient manner to the formation of gas bubbles, develops through electrolytic induction, whereas, in case of an anodic switching of the post part, depending on the composition of the treatment liquid, chlorine gas, oxygen, nitrogen, carbon monoxide and/or carbon dioxide develop. The gas bubbles forming thereby rise in the surrounding liquid and thus generate entraining effects, through which the above-mentioned surface components are also removed and discharged towards the outside. It was, for example, most surprisingly observed that, when using a solution containing positive ions, for example, an aqueous saline solution, these ions deposit on the post part 2 when the latter is cathodically switched and, thus, clearly increase the formation of gas bubbles. For example, the presence of Na+ ions in case of a cathodic switching of the post part 2 leads to a considerable formation of gas bubbles, because Na⁺ reacts at the cathode with the surrounding water and forms NaOH, releasing hydrogen.

In a third independent inventive aspect, also both with regard to the configuration of the system and with regard to the provided steps of the treatment method, the treatment element 30 is designed for a particularly simple and efficient combination of the before-mentioned aspects. This is based on the concept that both the provided feeding of the cleaning liquid and the specific detachment of the residues and fragments of bacteria and germs can be achieved by applying the above-mentioned energization in a common system and, thus, with particularly simple means.

The treatment liquid according to the invention is suitably chosen and composed in view of these aspects. Choice and composition of the basic constituents of the treatment liquid are chosen in particular in view of the intended function, i.e. application of an electric current in the space area of the surface needing treatment, it being in particular ensured that the electric conductivity of the treatment liquid is sufficiently high for this purpose. This shall be ensured in particular by a chosen sufficiently high ion density in the treatment liquid. For this purpose, a metallic salt, preferably in aqueous solution, is provided as a basic constituent of the treatment liquid. Said metallic salt supplies the ions for the transport of current and, in addition, the conversion products arising after the respective electrode reaction can also posses suitable biochemical effects. By specifically choosing a sufficiently high electric conductivity, it shall be ensured that during the performance of the cleaning method at the inserted implant the current flows through the treatment liquid and, thus, through the parts and components needing treatment, but not through the patient's body tissue, so that a risk for the patient through an unwanted current flow through soft tissue, bones, blood, and/or other body materials can be minimized. The electric conductivity of the treatment liquid should, if possible, amount to a multiple of the electric conductivity of blood, bones, soft tissue, fatty tissue, or other body materials.

Consequently, the following conductivity values are in particular taken into consideration in the choice and composition of the basic constituents of the treatment liquid (the electric conductivity a being indicated in the usual unit mS/cm):

Skin: 0.03-0.1 mS/cm
Bone: 0.06-0.2 mS/cm
Fatty tissue: 0.20-1.0 mS/cm
Muscular tissue: 0.80-2.5 mS/cm
Blood: approx. 6.7 mS/cm
Other body liquids: approx. 15 mS/cm

To keep the risk potential for the patient suitably low and to restrict the current flow to the desired regions, the electric conductivity should, therefore, amount to at least twice, preferably five times, particularly preferably ten times the conductivity of other body liquids. Therefore, the electric conductivity of the treatment liquid should have a value of at least 30 mS/cm, preferably at least 75 mS/cm and particularly preferably at least 150 mS/cm. In comparison with blood, this means that the electric conductivity of the treatment liquid preferably amounts to at least approx. five times, preferably at least approx. ten times and particularly preferably at least approx. twenty times the conductivity of blood. Measurements have shown that, when applying a treatment liquid chosen in this way, the electric voltage to which the body tissue, the blood, the body liquids, etc. are exposed, is lower than 6 V, preferably lower than 3 V, particularly preferably lower than 1.5 V, so that damages for the patient can securely be excluded, as the voltages are kept low. To achieve such a conductivity, in particular the ion concentration in the treatment liquid and in the basic constituents forming the latter are chosen sufficiently high; for this purpose, caustic solutions, acids, salts, and/or other ion-forming substances or compositions of matter can be used.

Choice and composition of the basic constituents of the treatment liquid take into consideration to a particularly high degree that the cleaning or biofilm-detaching effect of the electrolytic treatment of a contaminated implant surface is based on a combination of several causes, which should be made use of, if possible, complementarily to each other. On the one hand, gases or gas bubbles may form, when the current flows through the electrolyte, preferably in the area of the electrodes, which gases or gas bubbles have a detaching (mechanical) effect on the biofilm. These gases develop immediately at the implant surface serving as an electrode and, thus, between said implant surface and the biofilm. The growth rate and maximum size of the developing gas bubbles influence the detachment process.

The second reason for the implant-cleaning or biofilm-detaching effect of the electrolytic process is the decomposing, destroying, and dissolving effect of the electrolytically created substances or compositions of matter on the adhesion of the biofilm on the implant surface, i.e. on the gluing or anchoring mechanism.

The third reason for the cleaning or detaching effect of the electrolytic process is based on material-eroding effects of the implant material, through which component parts or particles of the implant properly speaking are extracted thereform in its surface area.

The fourth reason for the cleaning or detaching effect of the electrolytic process is based on the formation of an oxide layer of metallic implants, which allow this. In this case, metal atoms of the metallic basic material penetrate the possibly already existing oxide layer due to the applied electric voltage and react with substances of the electrolyte (mostly oxygen=>formation of metal oxide). In metals which do not form an oxide layer or do not form a mechanically stable oxide layer, non-oxidic compositions of matter (mostly salts) may also arise, which then get into solution.

The basic constituents provided for forming the treatment liquid are suitably chosen and combined with each other in view of these effects. Furthermore, it is taken into account as a fundamental design target that no toxic effects or effects which are hazardous or disagreeable to a patient in another manner should occur, so that the treatment liquid is also suitably for being applied on the inserted dental implant, i.e. in the patient's mouth. In the exemplary embodiment, the basic constituents provided are at least one salt, on the one hand, and one acid, on the other hand, preferably diluted with water, whose choice and composition depends in particular on the above-mentioned criteria. It is particularly preferable to provide, as an acid, phosphoric acid, citric acid, malic acid, ethanoic acid, lactic acid, carbonic acid, or a combination thereof. Alternatively or additionally, it is particularly preferable to provide, as a salt, sodium, calcium, aluminium, magnesium, or potassium iodide, chloride, nitrate, carbonate, or hydrogen carbonate, and/or ammonium chlorite, nitrate, or iodide, or a combination thereof.

Furthermore, it is taken into account that the provided electrolytic process can be guided, at choice, with anodic or with cathodic switching of the post part. Consequently, a difference is made in the following between an anodic reaction and a cathodic reaction.

In an anodic reaction, i.e. in case of an anodic switching of the post part 2, the anions present in the treatment liquid are oxidized on the anode, in general through the extraction of electrons. This may lead to an immediate reaction with the material, in particular to the formation of an oxide layer and/or of a salt, with the implant material. Bone implants and, thus, also the post part 2, mostly consist of titanium, zirconium, tantalum, or alloys of these metals. Furthermore, other metals are added by alloying. These metals or metal alloys possess in most cases a high degree of oxide-layer formation. This oxide-layer formation has a passivating effect on the surface, with the consequence that the anodic reaction of these metals or metal alloys is prevented or at least very greatly reduced. As in most cases, compositions of matter with oxygen are found in the biofilm, it is in most cases not possible to prevent this passivation. If the post part is switched anodically, the detaching and cleaning effect is, therefore, in most cases limited to the oxide-layer formation. It could be shown in extensive examinations that with higher operating voltages of, for example, more than 10 V, a material-eroding process is possible, but that the latter entails a strong heat development. This heat development may lead to an undesired necrosis of the bone. Furthermore, the material erosion resulting therefrom changes the properties of the original implant surface in an unwanted manner.

It has surprisingly turned out that, as an exception thereto, a basic material of the post part 2 containing aluminium as an alloy component (for example, titanium grade V, containing approx. 6% aluminium and 4% vanadium) enables an anodic energization of the post part 2, without the formation of an oxide layer excessively impeding the process. In this way, it is possible, depending on the composition of the treatment liquid, to generate chlorine or iodine gas or else CO₂ directly on the surface of the post part 2 and, thus, make it immediately usable for the intended detachment of the biofilm. For such a process guidance, the treatment element 30 is particularly advantageously provided with a conductive surface coating, for example made of DLC (“diamond-like carbon”), a metal, a conductive synthetic material, or an electrically conductive ceramic.

It has turned out to be particularly advantageous that a basic material of titanium grade IV or titanium grade V of the post part, by adding CO₂ to the treatment liquid, enables a formation of CO₂, Cl and/or I, allowing a current flow of long duration, in spite of the formation of an oxide layer under anodic energization.

For the above-mentioned reasons, the post part 2 is, however, in general preferably switched cathodically during the treatment with the treatment liquid. In this case, positively charged ions (cations) wander to the surface of the post part 2. These ions can be in particular H⁺ ions, metal ions or long-chain hydrocarbon ions, e.g. from ionic liquids. The salt provided as a basic constituent for the treatment liquid is in this case particularly purposefully chosen in view of the properties of the cations which shall promote the above-mentioned process or make it possible in the first place. To generate as high an electric conductivity as possible, small ions (H⁺ ions or metal cations) are particularly suitable, which, in addition, in the manner of another particularly favorable effect, are able, in a relatively easy manner, to penetrate the possibly existing biofilm. H⁺ ions are reduced to elementary hydrogen H on the cathode formed by the post part 2. This generates a formation of bubbles.

Alkali metals, alkaline earth metals and/or aluminium react on the cathode with the surrounding water and form elementary hydrogen and its metal cations and OH⁻ ions. This means that hydrogen bubbles and the hydroxide of the used metal ions form. Through the combination of these components, it is, therefore, achieved, in addition to the detaching effect of the arising hydrogen, that the metal hydroxide has an antibacterial effect and a diluting or dissolving influence on the biofilm or the latter's adhesion mechanism.

To avoid incompatibilities with the body tissue, in particular the metal cations produced naturally in the body (e.g. potassium and/or sodium ions) are particularly preferred as metal cations. Furthermore, calcium, magnesium and/or aluminium ions are also suitable. The salt provided as a basic constituent for the treatment liquid is, therefore, particularly preferably a salt of these metals, in particular because these metal cations can anyhow exclusively be made available in the form of a salt, e.g. dissolved in water.

These metallic salts can be compounds of the above-mentioned metals with a suitable halogen, for example with sulphur, phosphor, nitrogen, fluorine, chlorine, iodine, bromine, hydrocarbon, oxygen, boron, or other nonmetals. The halogen is advantageously suitably chosen considering the principle “the larger the anion, the lower the electric conductivity” and in view of the generally desired high electric conductivity. Furthermore, preferably only substances influencing neither health nor the periimplantary tissue are taken into consideration as anion. Furthermore, it has to be taken into account that disagreeable smells or taste compounds are unwanted. For these reasons, sulphur anions or anions containing sulphur in combination with oxygen or other elements are considered as rather unsuitable. This also applies to fluorine, bromine, nitrogen, and boron ions, possibly also in combination with other elements.

In contrast to that, phosphates, phosphate ions and hydrogen phosphate ions mostly have hardly any detrimental effect or none at all. Chlorine ions or ions containing chlorine mostly have an antibacterial effect. Should the chlorine ion, however, be electrolytically oxided and be present in water in the elementary state, hydrochloric acid and hypochlorous acid will form. It is true that, in combination with the cathodically generated hydroxide, this would lead to a neutralization, but examinations have shown that the chlorine arising on the counterelectrode to the implant (anode) escapes from the electrolyte to a great extent in the form of gas. If it is not possible to suck off the chlorine completely during the treatment, severe cauterizations in the lungs and/or the mucous membranes may result. In this case, one has to balance whether the benefit for the patient or the latter's endangerment is greater.

With regard to the phosphates of aluminium, potassium, sodium, calcium, or magnesium, it must, furthermore, be noted that their dissolubility in water is so low that a sufficient electric conductivity of the electrolyte is not guaranteed (these phosphates are, however, very well suited as additives of the electrolyte for buffering the pH-value). Although chlorides of the four above-mentioned metals would have a sufficient dissolubility in water and a good cleaning and killing effect on the biofilm, they cannot be considered as the optimum. In case of nitrates and/or nitrites, an endangerment of the patient through the formation of NOₓ gases has to be expected. For this reason, the use of nitrites or nitrates is not advisable.

In view of the above-mentioned design targets, in particular for a particularly good compatibility for the patient, iodine is provided in a preferred embodiment as halogen. It is particularly advantageous that iodine salts of potassium and of sodium are naturally present in the human body. Through the oxidation of iodine ions on the anode, first of all elementary iodine develops, which can dissolve in a sodium-iodide/potassium-iodide solution. An iodine-potassium-iodide solution or an iodine-sodium-iodide solution will result thereby. Both solutions are strong disinfectants, which have proved themselves in human medicine.

Pure solutions of sodium iodide or potassium iodide or a mixture of the two entail, however, the possible disadvantage of the formation of sodium hydroxide and/or potassium hydroxide and the resulting increase of the pH-value. It could, in fact, quite generally, be considered as a problem of the above-mentioned formation of metal hydroxide that a metal hydroxide increases the pH-value of the electrolyte. Such an increased pH-value and the developing caustic solution of the dissolved metal hydroxide might have an undesired influence on the surrounding tissue in the patient's mouth and in particular, on the bone. Furthermore, adjacent teeth might be damaged. Furthermore, the formation of hydroxides might lead to their precipitation on the post part 2 or generally on the component part needing treatment, due to their very low water solubility, thus impeding the further current flow and, thus, the process as a whole. At best when using a calcium salt in the treatment liquid, the developing calcium hydroxide, which is present in the bone material, could be integrated into the bone; calcium is, therefore, a particularly preferable constituent of the salt. To compensate these undesired influences, the treatment liquid contains the acid as another basic constituent in the manner of a pH-buffer or pH-reducer.

The acid, for its part, is chosen, in the manner of a design criterion, in such a way that it does not endanger, if possible, the patient or the periimplantary tissue, but, on the one hand, neutralizes the hydroxide (and prevents, if possible, an increase of the pH-value to more than 7), whereby, on the other hand, the reaction products should serve for the actual target of cleaning the implant body and removing the biofilm. As mineral acids, phosphoric acids and/or phosphate acids are preferred for that purpose. For reasons of hazards to health and/or to the bone/tissue, their concentration should be limited to maximally 30% or preferably, 10% to 20%. A particularly preferable acid, which is also considered as a mineral acid and which has a particularly positive effect on the overall target of killing and cleaning, is, on the other hand, carbonic acid. The usable quantity of the latter is, however, limited through its relatively low solubility in water.

Contrary thereto, organic acids, similar to mineral acids, provide pH-value-reducing and hydroxide-neutralizing H⁺ ions. As, in addition, they do not produce any damages, or at most slight damages, in the tissue or in the patient as a whole, such organic acids are most particularly preferred as a basic constituent of the treatment liquid. Organic acids are, for example, alkane acids, fruit acids, carboxylic acids as well as hydroxy carbonic acids. α-hydroxy carbonic acids have turned out to be particularly suitable acids. In particular, the particularly preferable acids lactic acid, citric acid, and malic acid have no effects hazardous to health on the patient in general or on the periimplantary tissue. Especially on implants greatly covered and contaminated with a biofilm, on which tartar has also developed, a good cleaning success was achieved with relatively low dosages of ethanoic acid. Other acids, which have the cleaning as well as the bactericidal effect, but, for health reasons, are not harmless, would be fumaric acid, gluconic acid, glycolic acid, salicylic acid, mandelic acid, tartaric acid, oxalic acid, and formic acid.

When the hydroxide ion OH⁻ is neutralized with the corresponding H⁺ ion of an acid, the metallic salt of the acid of the corresponding metal hydroxide will additionally be produced. The intended use of the acid is, therefore, not only advantageous for buffering the pH-value, but, in addition, contributes to the conversion of the relatively little water-soluble hydroxide into relatively well water-soluble salts, thus preventing the precipitation of unwanted deposits, detrimental to the process, on the component part needing treatment. The above-mentioned salts are in particular used when combining the above-mentioned preferred materials, among other, also in the field of medicine. During the neutralization of the potassium, sodium and/or calcium hydroxide with lactic acid, potassium lactate (possessing a broad-spectrum antimicrobial effect), sodium lactate or calcium lactate arises. It, however, the produced hydroxides are neutralized with citric acid, citrates of potassium, sodium or calcium will arise. Especially in the case of sodium citrate, this is particularly advantageous, as it prevents blood coagulation. This is particularly advantageous, because blood escaping during the process and coagulating on the implant surface might impede the ion wandering to the implant surface and, thus, the continuation of the treatment process as a whole.

Contrary thereto, in case of a neutralization of the hydroxides with malic acid, malates of the respective cation arise, which also have favorable effects on the process. in case of a neutralization of the hydroxides with ethanoic acid, acetates of potassium, sodium and/or calcium arise, which also have a favorable effect on the process.

Lactates, citrates, malates, and/or acetates of potassium, sodium and/or calciums all possess an acid-regulating effect and are so compatible that according to the present EU regulations concerning food additives, their use is not subject to any quantitative limitation.

When using acids in the electrolyte in combination with iodides and/or chlorides of sodium, potassium, magnesium, aluminium, and/or calcium, it has surprisingly turned out in the electrolytic application that the direct reduction of the H⁺ ions influences the formation of bubbles so positively that the biofilm comes off clearly more quickly and better. At a high generation rate, a multitude of relatively small bubbles develop, which due to their relatively small size are able to detach the biofilm as a whole and not only locally from the surface underneath it. In this way, the biofilm is preferably detached as a whole or in relatively large coherent pieces instead of a multitude of smaller fragments, which entails a clearly improved cleaning effect.

Instead of metal cations, ammonium cations can also be used. In this case, there exists, however, the risk that in the electrolytic process, other ammonium compounds (e.g. ammonia) are generated. This constitutes a risk for the patient and is also perceived through a very disagreeable taste and smell.

It was observed in tests that the biofilm comes off partially in very small fragments or else in larger coherent pieces. The latter is preferred, because in this case, very favorable cleaning results can be achieved on relatively large areas. Examinations have also shown that the removal of the detached biofilm and/or its fragments is promoted by a formation of foam on the implant surface. It has turned out that it is favorable to apply, after the use of an electrolyte consisting of the above-described metal salts, acids and water, responsible in particular for killing and detaching, a second electrolyte, which shows in addition a formation of foam in the area of the cathode. Such a formation of foam can be achieved by preferably adding to the electrolyte another substance comprising at least three CH₂ chain links or at least one CH₂ chain link and at least one carbon ring compound. Here, e.g. oil and/or chlorhexidine can be used. Furthermore, ionic liquids, which preferably contain I⁻, Cl⁻ and/or OH⁻ ions, can also be used. As the organic cation share of an ionic liquid is under certain circumstances reduced on the implant surface and remains there, it is possible in a particularly favorable embodiment, to add bone-growth factors to this cation share.

If chlorides and iodides are mixed in the correct ratio, the disturbing formation of chloric gas can be avoided. At the anode, the following is generated:

2J+5Cl+6H₂O→10HCl+2HIO₃

This means that both hydrochloric acid and iodic acid are formed at the anode. These acids certainly have a strong antimicrobial effect and are also neutralized again when meeting with the cathodically produced hydroxide.

A most particularly preferred composition of the treatment liquid, which in the laboratory test showed particularly favorable cleaning properties, comprises an aqueous solution of sodium iodide (NaI) or potassium iodide (KI) in a mixing ratio of at least 5 g, preferably at least 10 g, particularly preferably at least 20 g of the salt per 30 ml liquid (i.e. water H₂O, possibly enriched with CO₂), reduced, by the addition of lactic acid, to a pH-value of approx. 2.7 to 2.9.

In the process guidance, a mean current density at the post part 2 or at the component part needing treatment of at least 50 mA/cm², advantageously of at least 100 mA/cm², particularly preferably of at least 250 mA/cm², is provided, this current density being referred to the outer surface of the post part 2 (i.e. without taking into accoung any surface-enlarging properties, such as, for example roughness or surface structure). For the detachment of the biofilm, a mean current density of 50 mA/cm² to 300 mA/cm², advantageously of 100 mA/cm² to 200 mA/cm², has turned out to be particularly favorable. For the removal of the biofilm fragments, the mean current density should preferably be increased to the range of 300 mA/cm² to 5,000 mA/cm² or particularly advantageous of 1,000 mA/cm² to 2,000 mA/cm².

The addition of H₂O₂ greatly reduces or prevents the bubbling effect at the cathode. A very strong formation of H₂O results, which can be used for rinsing the surface.

For specifically feeding the above-mentioned treatment liquid, which is considered as independently inventive, into the space area at the post part 2 needing treatment, the treatment element 30 possesses a construction which can be taken from the perspective view according to FIG. 5 and the representation in longitudinal section according to FIG. 6, the treatment element 30 being in each case represented in the condition mounted on the post part 2. The representations also show a space area 36, surrounding the post part 2 in the area of its external thread 6 in a ring-shaped manner, in the jawbone 38 afflicted by periimplantitis and infested with bacteria.

The treatment element 30 includes a base body 40, substantially designed as a body in the form of a cylindrical casing, whose end face 42 forming the contact surface 32 is placed onto the upper end face or end edge 34 of the post part 2. Furthermore, to increase the mechanical stability, a connection stud 43 is additionally moulded onto the base body 40, whose contour and geometrical parameters are adapted to the receiving duct 12 in the post part 2 and which can be pushed into the latter.

In the interior of the base body 40 and coaxially therewith, a central inner duct 44 is provided, in which a connecting screw 46 is guided. The thread 48 of the connecting screw 46 engages into the screw thread 20 provided inside the post part 2. Contrary to the connecting screw 18 provided for connecting the actual abutment 4 with the post part 2, the connecting screw 46 is not designed for a high mechanical load-bearing capacity and longevity of the produced screw connection; the connecting screw 46 is rather based on other desigh criteria, taking into consideration in particular the course of treatment explained in the following, wherein the connecting screw 46 and, with it, the post part 2, shall serve as electrode for the current impulses. Consequently, the connecting screw 46 is made of an electrically conductive material, in particular of a metal, such as, for example, titanium.

The treatment element 30 is designed for feeding a cleaning liquid, which, among others, may also have the effect of killing germs or bacteria, into the space area 36. For this purpose, the base body 40 is provided with a plurality of media ducts 50, which are connected, on the inlet side, with a supply or feeding system for the treatment liquid. In the exemplary embodiment, the media ducts 50 are formed by grooves 54 moulded into an annular body 52 surrounding the base body 40. The annular body 52 is pushed onto the base body 40, so that the grooves 54 are closed towards the inside by the outer casing of the base body 40 and, thus, form a duct system made up of the media ducts 50. Alternatively, the media ducts may, of course, also be moulded directly into the base body 40 in another manner.

In the immediate vicinity of the contact area of the end face 42 of the base body 40 with the end edge 34 of the post part 2, the duct system formed by the media ducts 50 includes a plurality of outlet openings 60, of which FIG. 6 shows only two, for better clarity. In the exemplary embodiment, each media duct 50 is provided with an outlet opening 60. Cross-section and number of the outlet openings 60 can, however, also be adapted to individual specifications. For example, a single outlet opening might be provided, forming, for example, an annular gap on the entire periphery between the end face 42 and the end edge 34. Alternatively, a plurality of outlet openings 60 may be provided, which may be arranged uniformly around the base body 40, in particular viewed in the peripheral direction of the base body 40. The outlet openings 60 of the duct system formed by the media ducts 50 exit in the immediate vicinity of the end face 42 and, thus, immediately above the space area 36, so that medium flowing out of the outlet openings 60 gets more or less directly into the space area 36 situated therebelow. Through this embodiment of the base body 40, which is considered as such as an independent inventive aspect, the treatment element 30 thus forms a duct system, with which the treatment liquid can be introduced, in a purposeful and efficient manner, directly into the space area 36 needing treatment.

In addition, the treatment element 30 is also specifically configured as an electric system. As a design principle, it is in particular provided to make it possible to charge the medium carried in the media ducts 50, in particular the treatment liquid carried therein, with current impulses in a pulsed manner. The treatment element 30 is designed for producing the current flow provided for the purpose of cleaning the inserted implant part 2 in a specifically localized manner in the space area 36 needing treatment. The treatment element 30 is configured according to the design principle that the electric current is fed to the inserted implant part 2 and that the latter can be used as electrode. For this purpose, the treatment element 30 comprises a first conduction element 62, forming an electric current path and being electrically connected via the connecting screw 46 with the implant part 2, which, in turn, can be connected to a suitably chosen current or voltage source.

To form an opposite pole or the counterelectrode, it is provided to utilize the electric conductivity of the treatment liquid carried in the media ducts 50. For this purpose, the interior of the media ducts 50 is, in turn, electrically connected with the other pole of the current or voltage source. Thus, the outlet openings 60 of the media ducts 50 form in electric terms a contact 64 or an electric contact point, via which the current flow into, or out of, the implant part 2 is effected. With this utilization of the outlet openings 60, positioned in the immediate vicinity of the space area 36 needing treatment, as an electric contact 64, it is achieved that the electric current applied for the purpose of treatment and cleaning can flow through the surface zone of the inserted implant part 2 afflicted by the bacteria and, from there, as directly as possible, i.e. in particular without making any “detours” through further body tissue or the like, to the contact surface 64 or the contact point. Therefore, the media ducts 50, inclusive of the electrically conductive treatment liquid carried therein and the corresponding connection elements, form in the exemplary embodiment a second conduction element 66, forming an electric current path to the contact 64 arranged on the end side.

Alternatively, however, the second conduction element 66 could also be designed in the manner of a “conventional” electrode, i.e. in particular as an electrically conductive needle-like element made of metal. This electrode could in particular be mounted on the base body 40 so as to be shiftable in a longitudinal direction substantially parallel to the central axis of the base body 40. To form this electrode or an additionally provided third electrode, as required, which can be provided, for example, for locally generating an electric field, for example for strengthening of the field, a suitably shaped further metallic body 68 may additionally be provided. The treatment element 30 can also be designed without the media ducts, it being possible that the counterelectrode and, thus, the second current path, are formed exclusively by means of the metallic body 68. In this case, the contact 64 is formed by the end-side free surface of the respective electrode body.

The positioning of the outlet openings 60 and/or the end-side contact surface 69 of the metallic body 68 ensures, furthermore, that the contact surface 64 of the second conduction element 66 formed by them is positioned at a distance of at least 1 mm and of maximally 10 mm from the central longitudinal axis of the dental-implant part 2, viewed in lateral direction.

The base body 40 of the treatment element 30 can be made of an insulating material, such as, for example, a ceramic or synthetic material. In the exemplary embodiment, it is, however, made of metal, namely titanium. To guarantee a reliable electric insulation of the components against each other, its end face 42 forming the contact surface to the dental-implant part 2 is provided with an insulating coating 70 and, thus, configured in an electrically insulated manner. Furthermore, the annular body 52 is made of an insulating material, such as, for example, a ceramic.

In an alternative embodiment, the treatment element 30′, as shown in FIG. 7 in a perspective view, is provided with another duct system, which may be provided, for example, as a return duct for the treatment liquid, as a separate feeding line for introducing a media mixture, or else as a suction duct. For this purpose, the annular body 52 is in this embodiment surrounded by another annular body 71, into which also grooves 74 are moulded on the inside for forming additional media ducts 72.

In the above-explained embodiments, the media ducts 50 and/or the conduction elements 60, 66 are designed in a substantially integrated construction and guided inside the base body 40 or inside the annular body 52, 71 connected with the latter. Alternatively or additionally, however, some or all of the media ducts 50 and/or the conduction elements 60, 66 can also be arranged on the outside of the base body 40 and connected with the latter via suitable holding elements. This configuration is shown in the exemplary embodiment in a perspective view according to FIG. 8 and in a crosssectional view according to FIG. 9. In addition to the already explained components, the treatment element 30″ shown there is provided with duct elements 80 arranged on the outside of the annular body 52 so as to be shiftable in longitudinal direction. Said duct elements 80 can be designed, analogously to the media ducts 50, in the manner of hollow needles or the like, and can be charged with treatment liquid and can additionally serve as a conduction element 66. Alternatively, however, they can also be designed metallically in the manner of electrodes and connected in an electrically suitable manner with the current or voltage source. In addition, the exemplary embodiment according to FIG. 8 shows a variant, in which, in addition to the media ducts formed by the externally arranged duct elements 80, integrated media ducts 50, formed by grooves 54 in the annular body 52, are also provided.

The treatment element 30, 30′, 30″ is preferably used in a treatment system 90, as shown in FIG. 10. The treatment system 90 is provided for an inserted dental-implant part or post part 2 and comprises the treatment element 30, 30′, 30″ and, in addition thereto, a connection element 92 between the treatment element 30, 30′, 30″ and a hose package 94, a plug-in connection 96 between hose package 94 and of a supply and control unit 98 arranged outside the patient's mouth. This supply and control unit 98 contains a electric supply, which is able to apply a voltage and/or make a current flow between the electrode in the post part 2 and another electrode, which may be situated in the treatment element 30, 30′, 30″, the plug-in connections 96, the hose package 94 and/or the supply and control unit 98.

This voltage or current can be applied to the two electrodes as a direct voltage/current, with the polarity in both directions, or as an alternating voltage. If the voltage is an alternating voltage, it can have the shape of a sine, a triangle, a rectangle or any imaginable superimposition of these shapes, with different frequences. Furthermore, this alternating voltage can be superimposed by a direct voltage. It is also possible to use a pulsating direct voltage. To generate an electric field, a third, electrically insulated electrode can be provided, preferably accommodated in the treatment element 30, 30′, 30″.

As described above, it is particularly advantageous to feed several electrolytes differing from one another in composition either one after another or simultaneously to or onto the implant. The treatment system 90 is suitably configured for that purpose. In particular, the supply and control unit 98 contains reservoirs for at least two liquids or electrolytes, which can be conveyed into the treatment element 30, 30′, 30″ via pumps and via one or several valves or valve units simultaneously (mixingly) or one after another via the hose package 94. In a particularly favorable case, the supply and control unit 98 also contains a suction device, with which the liquids or electrolytes fed via the treatment element 30, 30′, 30″ can be sucked off after use. In a particularly favorable embodiment, the supply and control unit 98 also contains a CO₂ processing device for water or other liquids/electrolytes. For optimizing the process, a media temperature control can also be integrated into the supply and control unit 98.

The hose package 94 and the plug-in connections 96 are designed such that they are able to guarantee the current flow and the media flow. A complete equipment would in particular comprise three electric ducts and two liquid/electrolyte ducts.

The electrodes may be made of the same material as the post part 2. As the post parts 2 are preferably made of titanium or a titanium alloy, it is preferred to make the other electrode(s) of another metal. Titanium and metals similar to titanium mostly form a protective oxide layer acting as an insulator when anodically energized. In order not to limit the current flow through such an oxide layer, in case of a cathodic energization of the post part 2, it is advantageous to use, as a counterelectrode, a metal which forms hardly any oxide layer or none at all. In a particularly favorable case, this electrode corrodes neither through contact with the media/electrolytes nor under application of a voltage or current. Preferably, this electrode is made of gold, platinum, palladium.

Should the interior of the inserted implant/post part 2 also be contaminated and, consequently, be cleaned, it is possible to rinse the interior with the medium and charge it with current separately or together.

The conduction elements may also be designed in the form of a flexible or firm diaphragm, which does not allow any liquids to pass, but only the ions present in the electrolyte. In such an embodiment, preferably one of the current paths exits in the interior of the post part 2 and continues past the contact surfaces 32 which, in this case, seal only partially or not at all, up to the outer surface of the post part 2.

### LIST OF REFERENCE NUMBERS

1 Dental-implant system
2 First implant part/post part
4 Second implant part
6 External thread
8 Apical end
10 Connection stud
12 Receiving duct
14 Indexing element
16 Duct end piece
18 Connecting screw
20 Screw thread
30, 30′, 30″ Treatment element/treatment abutment
32 Contact surface
34 End edge
36 Space area
40 Base body
42 End face
43 Connection stud
44 Guide sleeve
45 Spacer blocks
46 Connecting screw
48 Screw thread
50 Duct/media duct
52 Annular body
60 Outlet opening
62 Conduction element
64 Contact
66 Conduction element
68 Metallic body
69 Contact surface
70 Insulating coating
71 Annular body
72 Media duct
74 Groove
90 Treatment system
92 Connection element
94 Hose package
96, 98 Supply and control unit

1. A method for removing a biofilm from a component, which comprises contacting a treatment liquid composed of an aqueous solution of an acid which is provided with a metallic salt in such a manner that a conductivity of at least 30 mS/cm results with the component.

2. The method of claim 1, in which the conductivity of the treatment liquid is at least 75 mS/cm, particularly preferably at least 150 mS/cm.

3. The method of claim 1, in which for the treatment liquid a salt of aluminum, of an alkali metal or of an alkaline earth metal is provided as metallic salt.

4. The method of claim 1, in which for the treatment liquid a potassium, sodium, calcium, magnesium, or aluminum salt is provided as metallic salt.

5. The method of claim 1, in which for the treatment liquid iodine is provided as a halogen for the metallic salt.

6. The method of claim 1, in which for the treatment liquid an organic acid is provided as acid.

7. The method of claim 1, in which for the treatment liquid an α-hydroxy carbonic acid, preferably lactic acid, citric acid, ethanoic acid or malic acid, or a combination of these constituents is provided as acid.

8. The method of claim 1, in which the treatment liquid has a pH-value of less than 5, preferably of less than 4, particularly preferably of approx. 2.7 to 2.9.

9. A treatment liquid for cleaning bacterially contaminated surfaces of bone implants, dental implants or other components contaminated with a biofilm, including an aqueous solution of a phosphate of potassium, sodium, calcium, magnesium, or aluminum, and/or an acid, in which the aqueous solution is provided with a metallic salt, which is composed of iodine as a halogen, in such a manner that a conductivity of at least 30 mS/cm results.

10. The treatment liquid of claim 9, whose conductivity is at least 75 mS/cm, particularly preferably at least 150 mS/cm.

11. The treatment liquid of claim 9, wherein a salt of aluminum, of an alkali metal or of an alkaline earth metal is provided as metallic salt.

12. The treatment liquid of claim 9, wherein a potassium, sodium, calcium, magnesium, or aluminum salt is provided as metallic salt.

13. The treatment liquid of claim 9, whose acid is an organic acid.

14. The treatment liquid of claim 9, whose acid is an α-hydroxy carbonic acid, preferably lactic acid, citric acid, ethanoic acid or malic acid, or a combination of these constituents.

15. The treatment liquid of claim 9, which has a pH-value of less than 5, preferably of less than 4, particularly preferably of approx. 2.7 to 2.9.

16. The method of claim 1, wherein the component is a bone implant or a dental implant.
